(19) United States
(12) Patent Application Publication
(10) Pub. No.: US 2006/0052659 A1
(43) Pub. Date: Mar. 9, 2006

(54) Cardiac device and method

(76) Inventors: Stephen R. Topaz, St. Helens, OR (US); Peter A. Topaz, Portland, OR (US); Demin Shen, Bandung (ID)

Correspondence Address: Ingrid McTaggart, 1816 S.E. 54th Avenue, Portland, OR, 97215, US

(21) Appl. No.: 10/935,500

(22) Filed: Sep. 7, 2004

(51) Int. Cl. A61N 1/362

(52) U.S. Cl. 600/16; 623/3.1

(57) ABSTRACT

One embodiment of a cardiac system may include a body that defines a first chamber and a second chamber therein, and structure for transferring pressure from said second chamber to the first chamber.

### BACKGROUND

Cardiac devices may be implanted in live beings, such as human patients, to support a weak vascular system and to exercise the heart of the patient after stem cells have been implanted into the cardiac region of the patient.

### BRIEF DESCRIPTION OF THE DRAWINGS

FIG. 1 is a schematic view of one embodiment of a cardiac device of the present invention implanted in a human patient.

FIG. 2 is a cross-sectional side view of one embodiment of a cardiac device in an unpressurized condition.

FIG. 3 is a cross-sectional side view of one embodiment of a cardiac device in a pressurized condition.

FIG. 4 is a top view of one embodiment of a cardiac device.

FIG. 5 is a graph of one embodiment of a pressure condition pattern of one embodiment of a cardiac device.

FIG. 6 is a graph of one embodiment of a pressure condition pattern of one embodiment of a cardiac device.

FIG. 7 is a perspective view of one embodiment of a mold for making one embodiment of a membrane of a cardiac device.

### DETAILED DESCRIPTION OF THE DRAWINGS

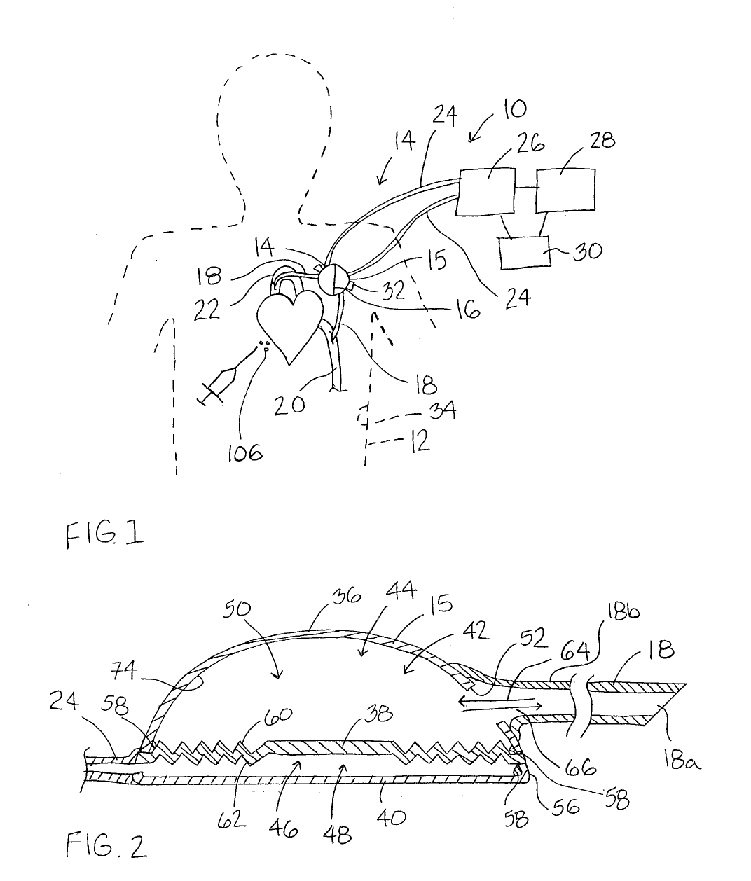

FIG. 1 is a schematic view of one embodiment of a cardiac system 10 of the present invention implanted in a human patient 12 (shown in environmental dash lines). In other embodiments patient 12 may comprise any living being, such as a domesticated or an undomesticated animal. Cardiac system 10 may include two separate cardiac assemblies 14, wherein each of cardiac assemblies 14 may include a cardiac device 15. Two cardiac devices 15 may be secured together by a fastener 16, such as mating hook and pile material. Cardiac devices 15 may each include a single connection structure 18 that may be placed into fluid contact with a blood vessel, such as the aorta 20 or the pulmonary artery 22 of patient 12. Cardiac devices 15 may each further include a connection structure 24 that may be operatively connected to a driver 26 which in turn may be connected to a controller 28. Controller 28 and driver 26 may be connected to a power device 30, such as a battery. Driver 26, controller 28, and battery 30 may be positioned interior or exterior (shown), or any combination thereof, of patient 12. Driver 26 may be a pneumatic driver and connection structure 24 may comprise a tube pneumatically connecting cardiac device 15 with driver 26. Connection structure 18 may comprise a cannula, as will be described in more detail below. Device 15 may further include an outwardly extending tab 32, or multiple tabs, that may be secured to a chest wall 34 of patient 12 to secure device 15 in place therein. In one embodiment tab 32 may be manufactured or DACRON®, or another adhesion promoting material, which may increase adhesion of tissue growth to tab 32 once device 15 is implanted. Driver 26, controller 28, battery 30 and cardiac device 15 may together comprise cardiac assembly 14, and two cardiac assemblies 14 may comprises cardiac system 12.

FIG. 2 is a cross-sectional side view of one embodiment of cardiac device 15 in an unpressurized condition. Cardiac device 15 may include a first member 36, a second member 38 and a third member 40. First member 36 may be referred to as a first body wall 36, second member 38 may be referred to a separation membrane 38, such as a flexible diaphragm 38, and third member 40 may be referred to as a second body wall 40. First body wall 36 and flexible diaphragm 38 may define a first chamber 42 therebetween that may define a first interior 44, and second body wall 40 and flexible diaphragm 38 may define a second chamber 46 therebetween that may define a second interior 48. Flexible diaphragm 38 may define an air-tight seal between first and second chambers 42 and 46 such that the chambers are separate from one another.

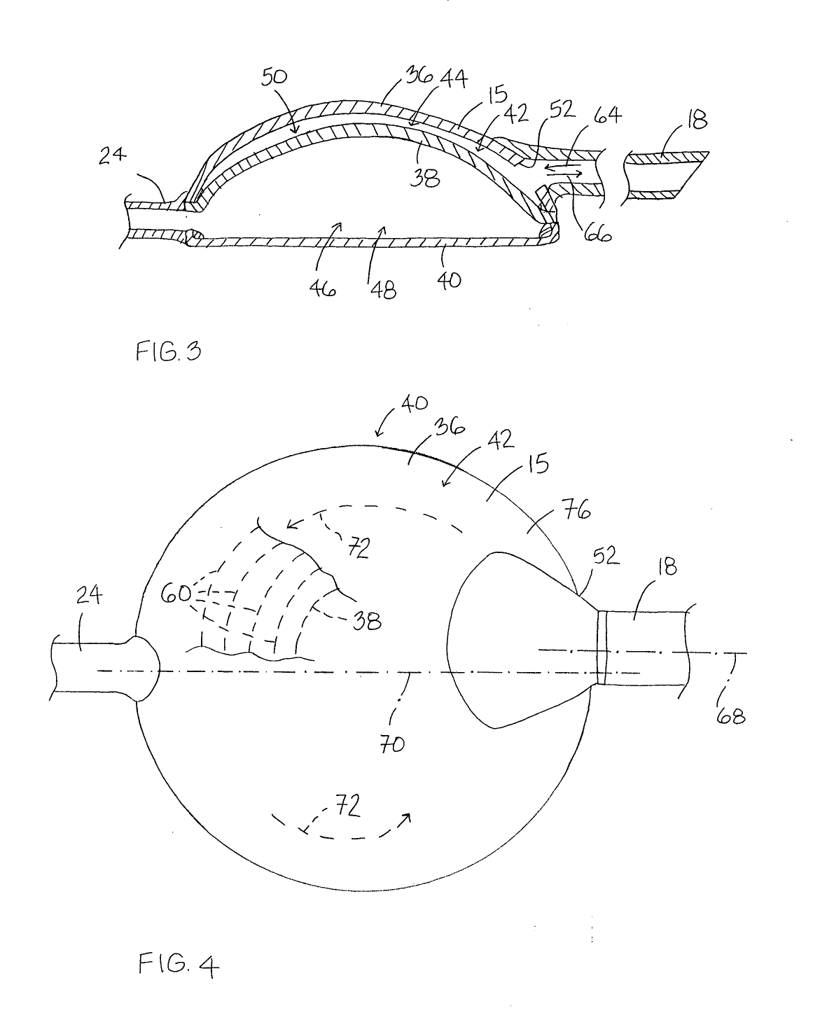

First and second interiors 44 and 48 together may define an interior 50 of device 15. The size of interior 50 may be constant whereas flexible diaphragm 38 may move from a first position (shown in FIG. 2) generally adjacent second body wall 40 to a second position (see FIG. 3) generally adjacent first body wall 36 such that the size of interiors 44 and 48 may vary depending on the position of flexible diaphragm 38. Accordingly, flexible diaphragm 38 in the first position may define an interior space of said first chamber 42 that may be larger than the interior space of second chamber 46, and flexible diaphragm 38 in the second position may define an interior space of first chamber 42 that may be smaller than the interior space of said second chamber 46. In one embodiment interior 50 may have a volume of approximately 100 cubic centimeters (cc). In another embodiment, interior 50 may have a volume of approximately 130 cc. However, device 15 may be manufactured in any size or shape so as to define any sized volume of interior 50.

Cannula 18 may be connected to a single opening 52 of first chamber 42 and connection tube 24 may be connected to a single opening 54 of second chamber 46. A single opening into a chamber may be described as the only access port for fluid or air flow into and out of the chamber. Accordingly, single opening 52 in first chamber 42 may be the only access port to first chamber 42 such that blood that flows through chamber 42 will enter and exit through single opening 52. Similarly, single opening 54 of second chamber 46 may be the only access port to second chamber 46 such that air that flows through chamber 46 will enter and exit through single opening 54. Moreover, openings 52 and 54 of device 15 may each define an unobstructed fluid flow having no valves or obstructions therein. Accordingly, flow into and out of the respective chamber 42 or 46 may be unrestricted by any mechanical or structure obstruction such that the flow through the chamber may only be subject to the pressure and/or volume constraints of the chamber.

Cannula 18 may include a first end region 18a that may be manufactured of a rigid material such that when end region 18a is inserted into a patient's aorta, blood flow may be directed away from the patient's head. A center region 18b of cannula 18 may be manufactured of a flexible material which may bend relative to the position of first chamber 42. Accordingly, by providing a flexible cannula 18 the physician implanting device 15 may be afforded a variety of possible positions in which to implant cardiac system 10, while still allowing proper placement of cannula 18 into the patient's aorta, or other blood vessel.

Connection tube 24 may be connected to driver 26 (see FIG. 1), which may comprise a pressurization device, such as a pneumatic driver. Controller 28 (see FIG. 1) may comprise a computer and may control driver 26 such that driver 26 may sequentially pressurize and depressurize interior 48 of second chamber 46. In the depressurized condition, shown in FIG. 2, flexible diaphragm 38 may be in an unbiased condition such that the flexible diaphragm 38 is positioned generally adjacent second body wall 40. In this condition, flexible diaphragm 38 may include alternating ridges 60 and grooves 62 and interior 44 of first chamber 42 may be approximately the same size as interior 50 of device 15. Due to the inclusion of flexibility structure, such as ridges 60 and grooves 62, flexible diagram 38 may be referred to as having a corrugated cross-sectional shape, an accordion cross-sectional shape and a folded cross-sectional shape.

Still referring to FIG. 2, in one embodiment, device 15 may be manufactured by creating each of top membrane 36, flexible membrane 38, and bottom membrane 40 on their own corresponding vacuum mold. The mold (see FIG. 7) may be manufactured of plastic. Such a vacuum mold process, utilizing a mold manufactured of rigid plastic, may allow the fabrication of device 15 in a timely and inexpensive manner when compared to the drip coating, metal-casted mold process of prior art devices. Each of membranes 36, 38 and 40 may be manufactured of flexible material, such as polyurethane.

To secure the membranes together, the three membranes may be simultaneously welded together along an edge region 56 of the membranes utilizing a radio frequency welder. In one embodiment, the radio frequency utilized may be a frequency of approximately 7.5 MHz. However, any frequency may be utilized, such as a frequency of approximately 40 MHz. After the three membranes are secured together along edge region 56 of device 15, a bead 58 of sealant, such as a polyurethane material, may be deposited along edge region 56 between top membrane 36 and diaphragm 38, and along edge region 56 between bottom membrane 40 and diaphragm 38. Beads 58 of sealant may be deposited in chambers 42 and 46 through openings 52 and 54, respectively. In this manner, first chamber 42 and second chamber 46 may each define an air-tight cavity.

FIG. 3 is a cross-sectional side view of one embodiment of cardiac device 15 in a pressurized condition wherein driver 26 may apply pneumatic pressure to second chamber 46 via connection tube 24. In the pressurized condition flexible diaphragm 38 may be in a biased condition such that the alternating ridges 60 (see FIG. 2) and grooves 62 (see FIG. 2) of flexible diaphragm 38 may be stretched and expanded and such that the flexible diaphragm may be positioned generally adjacent first body wall 36. In this condition, interior 44 of first chamber 42 may be very small and interior 48 of second chamber 46 may be approximately the same size as interior 50 of device 15. Driver 26 may sequentially pressurize and depressurize second chamber 46 such that first chamber 42 may be sequentially pressurized and depressurized by the action of flexible diaphragm 38. Flexible diaphragm 38, therefore, may define a pressure transfer structure that may transfer pressure from second chamber 46 to first chamber 42, while defining an air-tight seal therebetween.

During the pressurized state shown in FIG. 3, blood contained within first chamber 42 may be expelled from first chamber 42 in direction 64 through cannula 18 into aorta 20. The blood may be prevented from flowing into the patient's heart by the existing natural valves of the patient's heart. Moreover, the expulsion of blood from first chamber 42 may be timed by controller 28 to be just after the expulsion of blood from the patient's heart such that the blood from chamber 42 and the blood from the patient' heart both flow away from the patient's heart for circulation throughout the patient's body, at an increased pressure provided by device 15. During the depressurized state (see FIG. 2) blood contained with the heart (see FIG. 1) of the patient may be drawn in direction 66 from the patient's heart through the open valves of the patient's heart into first chamber 42 through cannula 18, at a low pressure provided by device 15. Accordingly, sequential operation of driver 26 may sequentially pressurize and depressurize second chamber 46 which in turn, by the action of flexible diaphragm 38, may sequentially pressurize and depressurize first chamber 42. Sequential pressurization and depressurization of first chamber 42 may sequentially pull blood from the patients heart into first chamber 42 through opening 18 and then expel the blood through opening 18 into the patients aorta. This sequential operation, therefore, may support or supplement a weak vascular system of the patient by simulating the pumping action of a healthy heart.

FIG. 4 is a top view of one embodiment of a cardiac device 15. In this embodiment, cannula 18 and opening 52 in first chamber 42 may be positioned along an axis 68 that may be non-contiguous with an elongate axis 70 of device 15. The off-axis positioning of cannula 18 and opening 52 may define a blood flow path 72 (shown in dash lines), such as a swirling flow path, within first chamber 42 that may reduce or eliminate stagnant flow points within chamber 42. Accordingly, the off-axis placement of cannula 18 and opening 52, with respect to elongate axis 70 of device 15, may reduce blood clotting within first chamber 42. Additionally, an interior surface 74 (see FIG. 2) of first chamber 42 and cannula 18 may be coated with a material, such as heparin that may inhibit blood clotting thereon.

An exterior surface 76 of device 15, tube 24 and cannula 18 may be coated with a material that may reduce or inhibit tissue growth thereto. In one embodiment exterior surface 76 of device 15 may be coated with heparin, or any other blood clotting inhibitor. Accordingly, in cases where device 15 may be implanted for a temporary time period, the device may be easily removed thereafter due to a limited amount of tissue growth to the device. In cases where device 15 may be permanently implanted into a patient, device 15 may not include a tissue growth inhibitor material coated thereon.

In this view, a portion of ridges 60 of diaphragm 38 are shown in dash lines to indicate the concentric nature of ridges 60 and grooves 62 on diaphragm 38 in this particular embodiment. Of course, other flexibility structure, and other orientations and sizes of ridges 60 and grooves 62 may be utilized in the cardiac system 10 of the present invention.

FIG. 5 is a graph of one embodiment of a pressure condition pattern of one embodiment of cardiac system 10 wherein cardiac assembly 14 may be utilized to maintain and supplement the vascular system of patient 12. Line 80 may represent the electrical signal/potential of the heart of patient 12. Line 82 may represent the normal blood pressure of a sick patient. Line 84 may represent the pressure applied to cardiac device 15, which may result in the sequential pressurization and depressurization of second chamber 46, and thereby the sequential pressurization and depressurization of first chamber 42. Line 86 may represent the cardiac device-assisted blood pressure of the patient, due to pressure pattern 84 applied to cardiac device 15. As shown in the figure, cardiac device-assisted blood pressure 86 may define a lower pressure than the normal blood pressure 82 of the patient in systolic region 78 when the patient's heart is contracting and expelling blood. Cardiac device-assisted blood pressure 86 may define a higher pressure than the normal blood pressure 82 of the patient in diastolic region 79 when the patient's heart is relaxing and blood is circulating through the patient's vascular system. Accordingly, device 15 may be utilized to lower the load upon the patient's heart while blood is expelled from the patient's heart. Device 15 may also be utilized to increase the pressure within the circulatory system after blood is expelled from the patient's heart. These two functions of device 15 support proper functioning of the patient's vascular system.

The sequential pressurization and depressurization of first chamber 42 may also be described as follows: Cardiac device 15 normally may perform two functions when supporting the normal bodily functions required for life. The first function may be to reduce the load of a patient's sick heart. This may be accomplished by assist device 15 removing blood from the outlet of the patient's heart, thereby lowering the load on the patient's heart, just as the patient's heart is delivering blood to the circulatory system. The second function of device 15 may be to generate enough blood at a high enough flow rate and pressure for the patient's body to operate normally.

The first function of device 15 may be accomplished by pneumatic driver 26 lowering the air pressure to cardiac assist device 15 to a point where blood in the outlet of the patient's heart is drawn into device 15. This removal of blood at a time that the patient's heart is ejecting blood may lower the patient's blood pressure to a level where the weakened patient's heart can push the required blood volume from their heart chamber into their circulatory system. This may be illustrated by region 78 of FIG. 5.

The second function of device 15 may be accomplished by pneumatic driver 26 raising the air pressure to device 15 to a level such that the blood that was drawn from the outlet of the patient's heart may now return to the patient's circulatory system. This reintroduction of the removed blood volume may raise the blood pressure in the patient's circulatory system to a level that the required flow and pressure functions of the patient's body can take place. This may be illustrated by region 79 of FIG. 5.

Still referring to FIG. 5, in the particular example illustrated, pressure 84 may be applied via driver 26 to second chamber 46 at or slightly before the end point 82a of the patient's normal systolic contraction of the patient's heart and pressure may be released from chamber 46 at or slightly before the beginning point 82b of the patient's normal systolic contraction of the patient's heart. In the embodiment shown, pressure 84 may have a high value 84a of approximately 2 to 3 pounds per square inch (psi) and a low value 84b of approximately −0.5 to −6.0 psi. Of course, other pressure values and sequences may be utilized as may be appropriate for a particular patient or condition. Moreover, controller 28 may be programmed to be dynamic in nature such that the pressure applied to second chamber 42, and the timed sequence of the applied pressure to chamber 42, may be adjusted in real time in response to internal conditions within the patient's body.

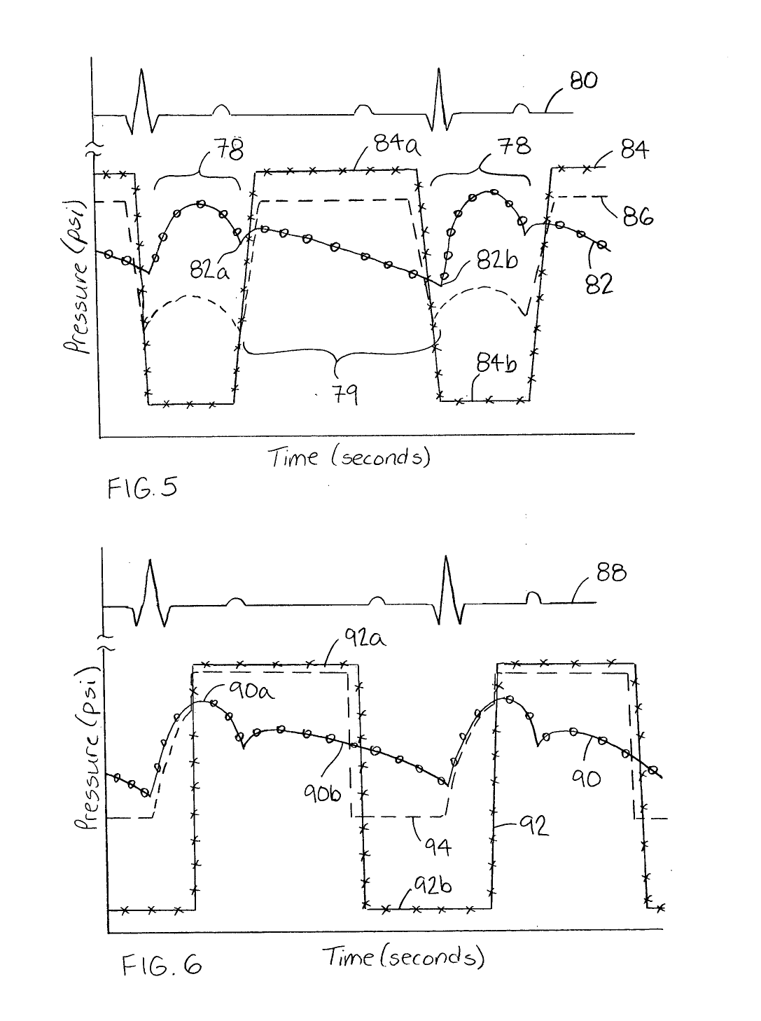

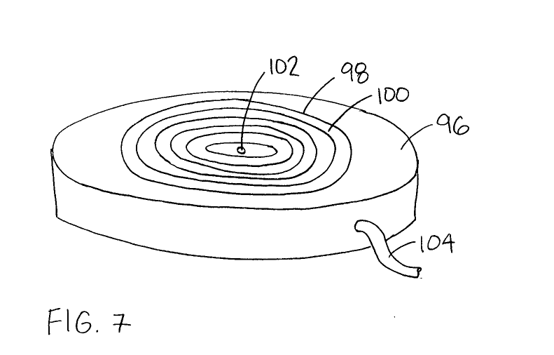

FIG. 6 is a graph of one embodiment of a pressure condition pattern of one embodiment of cardiac system 10 wherein cardiac assembly 14 may be utilized to exercise the heart of patient 12 after stem cells have been implanted into the patient's heart. Accordingly, this graph may represent one sequential control pattern of device 15 that may be utilized to repair the patient's heart. Line 88 may represent the electrical signal/potential of the heart of patient 12. Line 90 may represent the normal blood pressure of a sick patient. Line 92 may represent the pressure applied to cardiac device 15, which may result in the sequential pressurization and depressurization of second chamber 46, and thereby the sequential pressurization and depressurization of first chamber 42. Line 94 may represent the cardiac device-assisted blood pressure of the patient, due to the pressure pattern 92 applied to cardiac device 15.

The method of repairing cardiac damage in a live being may be described as follows. First, a cardiac system 10 may be implanted in a live being. The cardiac system 10 may include a first chamber 42 having a single blood flow port 18 connected to an aorta of a heart of the live being, such as a human patient. Stem cells 106 (see FIG. 1) may be added to the heart region, such as the heart muscle tissue, of the live being, wherein the stem cells may regenerate muscle tissue of the living heart. After implantation of the cardiac system 10 and the stem cells 106, the cardiac system may be sequentially pressurized and depressurized, such as by use of a pneumatic driver 26 and a controller 28, so as to sequentially push blood into the heart from the cardiac system, thereby “loading” or stressing the patient's heart with an added blood flow. The sequential pressurizing and depressurizing of the patient's heart may be conducted by sequentially pressuring second chamber 46 which may transfer the sequential pressure condition to first chamber 42. This may exercise the patient's heart thereby stimulating growth of the stem cells 106 and repair of the patient's heart 12.

More particularly, the sequential pressurization and depressurization of first chamber 42 may accomplish two functions when assisting in the healing of a patient's heart 12 that has had stem cells 106 introduced into the heart muscle. The first function may be to exercise the heart muscle to facilitate the changing of stem cells into functioning heart muscle cells. The cardiac exercise may be accomplished by loading the heart muscle as the patient's heart muscle is pumping blood into the patient's circulatory system. The loading may be accomplished by adding blood to the outlet of the patient's heart by pumping blood out of assist device 15 during the time when the patient's heart is pumping blood into the circulatory system. This pumping action of cardiac assist device 15 may be timed with the pumping action of the patient's heart by using the patient's electrocardiogram (EKG) or the patient's blood pressure to direct the pneumatic driver or the controller to change the gas driving pressure in an appropriate manner, as shown in FIG. 6.

The second function of the cardiac assist device 15 while exercising the patient's heart may be to maintain an adequate blood volume flow and average normal blood pressure for normal circulatory functions. Pneumatic driver 26 or controller 28 may receive electrical signals from the patient's EKG or from a blood pressure transducer and may change the driving gas pressure of driver 26 in a manner that would remove blood volume from the circulatory system, thereby creating normal average blood pressure and blood flow conditions in the patient's circulatory system.

Still referring to FIG. 6, in the particular example illustrated, pressure 92 may be applied via driver 26 to second chamber 46 at or slightly before the apex 90a of the patient's normal blood pressure peak during the systolic contraction of the patient's heart and pressure may released from chamber 46 at or slightly after approximately the midpoint 90b of the patient's normal diastolic relaxation of the patient's heart. In the embodiment shown, pressure 92 may have a high value 92a of approximately 2 to 3 psi and a low value 92b of approximately −0.5 to −6.0 psi. Of course, other pressure values and sequences may be utilized as may be appropriate for a particular patient or condition.

FIG. 7 shows one embodiment of a mold 96 that may be used to create the membranes, such as membranes 36, 38 and 40 (see FIG. 2), utilized in the fabrication of device 15 (see FIG. 1). In the embodiment shown, mold 96 may include ridges 98 and grooves 100 therein and may be utilized to fabricate flexible diaphram 38. Mold 96 may include a centrally located aperture 102 that may be connected to a vacuum device (not shown) via a conduit 104 so as to create a vacuum at aperture 102. When material is placed on mold 96 over aperture 102, a vacuum may be pulled on conduit 104 and on the material positioned on mold 96 such that vacuum mold 96 may be utilized to shape the material into a desired shape and configuration matching the shape and configuration of mold 96, as will be understood by those skilled in the art.

In the embodiment shown, device 15 may have a width in a range of 2 to 5 inches and a total height from the bottom of third membrane 40 to the top first membrane 36 in a range of 0.5 to 4 inches. Of course, device 15 may be manufactured in any shape or size as suited for a particular application.

Other variations and modifications of the concepts described herein may be utilized and fall within the scope of the claims below.

We claim:

1. A cardiac system, comprising:
a body including a first chamber and a second chamber separated by a flexible diaphragm;
said first chamber including a single opening to access an interior thereof; and
said second chamber including a single opening to access an interior thereof.

2. A cardiac system according to claim 1 wherein said body and said flexible diaphragm are manufactured of polyurethane.

3. A cardiac system according to claim 1 wherein said body is coated with a material that reduces tissue growth thereto.

4. A cardiac system according to claim 1 wherein said flexible diaphragm is corrugated.

5. A cardiac system according to claim 1 wherein said body defines an interior volume, wherein said diaphragm is movable to a first position such that said first chamber defines said volume, and said diaphragm is movable to a second position such that said second chamber defines said volume.

6. A cardiac system according to claim 1 further including a cannula connected to said single opening of said first chamber, said cannula adapted to be connected to a blood vessel.

7. A cardiac system according to claim 1 further including a tube connected to said single opening of said second chamber, said tube adapted to be connected to a pneumatic driver.

8. A cardiac system according to claim 7 further including a pneumatic driver connected to said tube, wherein said pneumatic driver sequentially pressurizes and depressurizes said second chamber.

9. A cardiac system according to claim 8 further including a controller adapted to control operation of said pneumatic driver.

10. A cardiac system according to claim 1 wherein said opening in said first chamber is positioned along an axis non-contiguous with a central axis of said body.

11. A cardiac system according to claim 1 further comprising a tab extending outwardly from said body, said tab adapted to be secured to an interior of a chest wall.

12. A cardiac system according to claim 6 wherein said opening and said cannula define an unobstructed blood flow path having an absence of valves therein.

13. A cardiac system according to claim 7 wherein said opening and said tube define an unobstructed pressurizing fluid flow path having an absence of valves therein.

14. A cardiac system according to claim 5 wherein said interior volume is at least 100 cubic centimeters.

15. A method of manufacturing a cardiac system, comprising:
forming a first body wall;
forming a second body wall;
forming a flexible diaphragm; and
welding said first body wall to said second body wall with said flexible diaphragm positioned therebetween, said welding comprising radio frequency welding.

16. A method according to claim 15 wherein said cardiac system defines a first chamber between said first body wall and said diaphragm and a second chamber between said second body wall and said diaphragm.

17. A method according to claim 15 wherein said radio frequency welding is conducted at a frequency of approximately 7.5 MHz.

18. A method according to claim 15 wherein said first body wall and said second body wall are flexible.

19. A method according to claim 15 further comprising adding a sealant solution along a seam between said first body wall and said flexible diaphragm and along a seam between said second body wall and said flexible diaphragm.

20. A method according to claim 15 further comprising securing a cannula to a single opening in said first body wall.

21. A method according to claim 15 further comprising securing a tube to a single opening in said second body wall.

22. A method according to claim 21 further comprising securing a pneumatic device to said tube, said pneumatic device operable to pressurize a space between said second body wall and said diaphragm so as to move said diaphragm toward said first body wall, and said pneumatic device operable to depressurize said space between said second body wall and said diaphragm so as to move said diaphragm toward said second body wall.

23. A method according to claim 15 wherein said cardiac system comprises a first chamber defined by said first body wall and said flexible diaphragm and a second chamber defined by said second body wall and said flexible diaphragm.

24. A method according to claim 15 further comprising making a first mold to form said first body wall, making a second mold to form said second body wall, and making a third mold to form said flexible diaphragm, wherein said first, second and third molds are manufactured of plastic.

25. A method according to claim 15 wherein said flexible diaphragm defines an accordion shaped cross-section.

26. A method according to claim 15 wherein said first body wall, said second body wall and said flexible diaphragm are each manufactured of flexible polyurethane.

27. A method according to claim 15 wherein said forming said first body wall comprises forming said first body wall on a vacuum mold, wherein said forming said second body wall comprises forming said second body wall on a vacuum mold, and wherein said forming said flexible diaphragm comprises forming said flexible diaphragm on a vacuum mold.

28. A cardiac system, comprising:
a body including a first chamber and a second chamber separated by a flexible diaphragm;
a pneumatic device connected to said second chamber wherein said pneumatic device is adapted to sequentially pressurize and depressurize said second chamber, wherein said first chamber is pressurized when said second chamber is depressurized and depressurized when said second chamber is pressurized; and
a cannula including a first end secured to said first chamber and a second end adapted to be connected to a blood vessel.

29. A cardiac system according to claim 28 wherein said flexible diaphragm in an unbiased condition includes alternating ridges and grooves.

30. A cardiac system according to claim 28 wherein said first chamber is accessed only by said pneumatic device and wherein said second chamber is accessed only by said cannula.

31. A cardiac system according to claim 28 wherein said first chamber is defined by a first wall and said flexible diaphragm, wherein said second chamber is defined by a second wall and said flexible diaphragm, and wherein when said first chamber is pressurized said flexible diaphragm is positioned adjacent said second body wall and when said second chamber is pressurized said flexible diaphragm is positioned adjacent said first body wall.

32. A cardiac system, comprising:
a body that defines a first chamber and a second chamber therein; and
structure for transferring pressure from said second chamber to said first chamber.

33. A cardiac system according to claim 32 wherein said structure for transferring pressure comprises a flexible diaphragm positioned within said body and between said first and second chambers.

34. A cardiac system according to claim 33 wherein said flexible diaphragm defines a sheet of material having a generally circular, concentric set of ridges and grooves.

35. A cardiac system according to claim 32 wherein said structure for transferring pressure is adapted for movement between a first position and a second position, wherein said structure in said first position defines an interior space of said first chamber that is larger than an interior space of said second chamber, and wherein said structure in said second position defines an interior space of said first chamber that is smaller than an interior space of said second chamber.

36. A cardiac system according to claim 32 wherein said first chamber is accessed through a single port, and said second chamber is accessed through a single port.

37. A method of circulating blood in a live body, comprising:
providing a man-made cardiac device including a first chamber and a second chamber separated by a flexible membrane;
connecting said first chamber to a live blood vessel;
connecting said second chamber to a pressurization device; and
sequentially pressurizing said second chamber between a first pressure condition and a second pressure condition, wherein said flexible membrane transfers said pressure condition of said second chamber to said first chamber such that during said first pressure condition blood is drawn into said first chamber and during said second pressure condition blood is expelled from said first chamber.

38. A method according to claim 37 wherein said second chamber includes a single access opening, and wherein said pressurization device is connected to said single access opening.

39. A method according to claim 37 wherein said first chamber includes a single access opening, and wherein said step of connecting said first chamber to a live blood vessel comprises connecting said single access opening to a live blood vessel.

40. A method according to claim 37 wherein said pressurization device comprises a pneumatic air pump.

41. A method according to claim 37 wherein said flexible membrane defines a fluid-tight seal between said first and second chambers, and wherein said flexible membrane is manufactured of polyurethane.

42. A method according to claim 37 wherein said flexible membrane in an unpressurized condition includes a plurality of folds therein.

43. A method according to claim 37 wherein said sequentially pressurizing and depressurizing said second chamber comprises pressurizing said second chamber approximately at an end point of the patient's normal systolic contraction of the patient's heart and depressurizing said second chamber at approximately the beginning point of the patient's normal systolic contraction of the patient's heart.

44. A method according to claim 37 wherein said pressurizing said second chamber comprises pressurizing said second chamber to a pressure of in a range of 2 to 3 psi, and said depressurizing said second chamber comprises lowering a pressure within said second chamber to a pressure in a range of −0.5 to −6.0 psi.

45. A method of implanting a cardiac device in a live being, comprising:
implanting a first chamber in said live being, said first chamber including a single access port defined by a cannula;
placing said cannula into fluid contact with a blood vessel of said live being;
implanting a second chamber in said live being, said second chamber being fluidly non-connected to said first chamber and pressurily connected to said first chamber so as to transfer a pressure condition of said second chamber to said first chamber.

46. A method according to claim 45 further comprising connecting said second chamber to a pressurization device.

47. A method according to claim 45 further comprising:
implanting a third chamber in said live being, said third chamber including a single access port defined by a cannula;
placing said cannula of said third chamber into fluid contact with a second blood vessel of said live being;
implanting a fourth chamber in said live being, said fourth chamber being fluidly non-connected to said third chamber and pressurily connected to said third chamber so as to transfer a pressure condition of said fourth chamber to said third chamber.

48. A method according to claim 47 wherein said blood vessel comprises an aorta and said second blood vessel comprises a pulmonary artery.

49. A method according to claim 47 wherein said first and second chambers define a first body including a first outwardly extending tab and said third and fourth chambers define a second body including a second outwardly extending tab, said method further comprising securing said first outwardly extending tab to a chest wall of said live being, and securing said second outwardly extending tab to a chest wall of said live being.

50. A method according to claim 45 wherein said cannula is placed into fluid contact with said blood vessel of said live being such that a blood flow path out of said first chamber extends away from a head of said live being.

51. A method of repairing cardiac damage in a live being, comprising:
implanting a cardiac device in a live being, said cardiac device including a chamber having a single blood flow port connected to a blood vessel of a heart of said live being;
adding stem cells to the heart of a live being; and
sequentially pressurizing and depressurizing said chamber so as to sequentially push blood into the live being's circulatory system from said chamber.

52. A method according to claim 51 wherein said cardiac device comprises a second chamber connected to said chamber by a flexible diaphragm that transfers a pressure condition of said second chamber to said chamber, and wherein said sequentially pressurizing and depressurizing said chamber comprises sequentially pressurizing and depressurizing said second chamber.

53. A method according to claim 51 wherein said sequentially pressurizing and depressurizing said chamber comprises utilizing a pneumatic driver operatively connected to said chamber.

54. A method according to claim 51 wherein said sequentially pressurizing and depressurizing said chamber is conducted according to the pressurization pattern shown as line 92 in FIG. 6.

55. A method according to claim 51 wherein said sequentially pressurizing and depressurizing said chamber comprises pressurizing said chamber approximately at an apex of the patient's normal blood pressure peak during a systolic contraction of the patient's heart, and pressurizing said chamber at approximately a midpoint of a diastolic relaxation of the patient's heart.

56. A method according to claim 51 wherein said pressurizing said chamber comprises pressurizing said chamber to a pressure in a range of 2 to 3 psi, and said depressurizing said chamber comprises lowering a pressure within said chamber to a pressure in a range of −0.5 to −6.0 psi.
